(19) United States
(12) Patent Application Publication
(10) Pub. No.: US 2009/0162306 A1
(43) Pub. Date: Jun. 25, 2009

(54) Topical composition comprising coloring antioxidants

(75) Inventors: Naresh Dhirajlal Ghatlia, Southbury, CT (US); Pushker Sona, Shanghai (CN); Endang Saraswati Angka Ang, Bangkok (TH)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc., d/b/a UNILEVER

(21) Appl. No.: 12/004,572

(22) Filed: Dec. 21, 2007

(51) Int. Cl. A61K 8/31; A61K 8/49; A61Q 1/02

(52) U.S. Cl. 424/63

(57) ABSTRACT

Topical compositions for delivering a desirable color are described. The color suitable to be delivered is stable and the result of colorants comprising an antioxidant like lycopene and a second colorant which is a dye or pigment. The topical compositions are stable notwithstanding the fact that they are free of formaldehyde generating preservatives.

### FIELD OF THE INVENTION

The present invention is directed to a topical composition comprising a colorant which is an antioxidant. More particularly, the invention is directed to a topical composition for influencing a desirable skin color where the topical composition comprises more than one colorant. A first colorant used is typically a dye or pigment, and the second colorant used is an antioxidant like a lipophilic carotenoid having a log P of greater than about 12.5. The topical composition, surprisingly, has an L* value from about 60 to about 90 and a hue value from about −50 to about 50 for at least about three (3) months and when stored at about ambient temperature. Moreover, such a composition is surprisingly stable in the absence of conventional preservatives that are known to release toxic compounds like formaldehyde.

### BACKGROUND OF THE INVENTION

Cosmetic compositions suitable for changing the appearance of keratinaceous substances like mammalian skin and nails are often desired. Women tend to prefer cosmetics that impart a matte appearance. The matte finish tends to overcome the shiny effect engendered by greasy skin and often creates a physical skin lightening effect generally associated with inorganic particulates. In fact, absorbent fillers like talc, silica, kaolin, and other inorganic particulates have been used to achieve such an effect by their optical properties. This effect is not always desired since many consumers feel traditional compositions are too white and create a non-natural look after application. Furthermore, consumers prefer to apply to their skin (especially their face) compositions that have natural ingredients. In fact, compositions with ingredients naturally found in skin are highly desired, as are compositions that have a desirable color which remains stable in the product.

There is an increasing interest to develop a topical composition that results in a desirable skin color after application. This invention, therefore, is directed to a topical composition for influencing a desirable skin color where the topical composition comprises more than one colorant. The composition comprises a first colorant which is a dye or pigment, and a second colorant that is an antioxidant like a lipophilic carotenoid having a log P of greater than about 12.5. The topical composition of this invention, surprisingly, is free of a brownish color often associated with oxidized carotenoids, and has a L* value from about 60 to about 90 and a hue value from about −50 to about 50 for at least about 3 months when stored at about ambient temperature. The topical composition of this invention is stable, notwithstanding the fact that it is substantially free of conventional preservatives known to release toxic compounds like formaldehyde.

### Additional Information

Efforts have been disclosed for making cosmetic compositions. In United States Published Application No. 2004/0071745, compositions with at least one carotenoid and at least one isoflavonoid are described, where the compositions are intended for treating the cutaneous signs of aging.

Other efforts have been disclosed for making cosmetic compositions. In U.S. Pat. No. 5,834,445, compositions with cyclodextrin-complexed beta-carotene and uncomplexed beta-carotene are described.

Still other efforts have been disclosed for making cosmetic compositions. In United States Published Application No. 2006/0171909, a cosmetic composition with an encapsulated dye is described.

None of the additional information above describes a topical composition comprising coloring antioxidants that are naturally found in skin, like a lipophilic carotenoid having a log P of greater than about 12.5. Particularly, none of the additional information describes a topical composition having a stable L* value from about 60 to about 90 and a hue value from about −50 to about 50 where the color of the composition is the result of a combination of at least one of a dye or pigment, and a lipophilic carotenoid having a log P of greater than about 12.5.

### SUMMARY OF THE INVENTION

In the first aspect, the present invention is directed to a composition for influencing skin color comprising:

(a) a first colorant comprising dye, pigment or both; and

(b) a second colorant;

the second colorant being an antioxidant and comprising a lipophilic carotenoid having a log P of greater than about 12.5 where the composition has a L* value from about 60 to about 90 and a hue value from about −50 to about 50 for at least about three (3) months when stored at about ambient temperature and further where the weight ratio of the first colorant (a) to second colorant (b) satisfies:

a/b=about 0.004 to about 1,000.

In a second aspect, the present invention is directed to a consumer grade colorant composition comprising:
(a) a first colorant comprising dye, pigment or both; and
(b) a second colorant comprising a lipophilic carotenoid having a log P of greater than about 12.5;
wherein the weight ratio of first colorant (a) to second colorant (b) satisfies

a/b=about 0.004 to about 1,000.

In a third aspect, the present invention is directed to a method for influencing the color of skin with the first aspect of this invention.

In an especially preferred aspect, the composition for influencing skin color and the colorant composition are substantially free of formaldehyde releasing preservatives.

Additional aspects of the present invention will more readily become apparent from the description and examples which follow.

The value L*, as used herein, means the CIELAB color coordinate for luminance or lightness. Hue is defined to mean [tan−1(b*/a*)] where a* is the CIELAB color coordinate for red/green and b* is the yellow/blue color coordinate. Substantially free of means less than about 0.015%, and preferably, zero to less than about 0.01% by weight, based on total weight of the composition for influencing skin color or consumer grade colorant composition.

Formaldehyde releasing preservative is meant to mean a preservative suitable to generate H₂CO when in an end use composition. Such preservatives include, for example, quarternium-15, imidozolidinyl urea, diazolidinyl urea, DMDM hydantoin, bromonitropropane diol or the like.

Skin, as used herein, is meant to mean skin on the face and body. The composition of this invention can be a base or sold as an end use composition. Such a composition may be sold in any consumable acceptable form such as a bar, liquid, stick, roll-on formulation, cream, fabric applied formulation, mousse, lotion, ointment cosmetic, or foundation.

### DETAILED DESCRIPTION OF THE PREFERRED EMBODIMENTS

The only limitations with respect to the dye and/or pigment that may be used in this invention is that the same is/are safe for topical application, compatible with a lipophilic carotenoid and suitable to be used to generate a composition with the color characteristics as defined herein. Such dyes and/or pigments are preferably those that are allowed in all cosmetic compositions, including those that may come in contact with lips.

Illustrative non-limiting examples of the types of dyes and/or pigments that may be used in this invention include Pigment Red 4, Food Red 1, Acid Orange 7, Pigment Red 57, Food Yellow 3, Acid Red 33, Food Red 12, Acid Yellow 23, Food Yellow 4, Food Green 3, Food Blue 2, Acid Blue 9, Solvent Red 43, Acid Red 87, Solvent Red 48, Acid Red 92, Solvent Violet 13, Acid Violet 43, Solvent Green 3, Acid Green 25, Vat Red 1, Pigment Metal 1, Pigment Blue 29, Pigment White 21, Pigment White 22, Pigment White 14, Pigment White 31, Pigment Brown 6, or Pigment Red 101, Pigment 102, Pigment Brown 6, Pigment Yellow 42, Pigment Yellow 43, Pigment Black 11, Pigment White 18, Pigment Violet 16, Pigment White 6, Pigment White 4, mixtures thereof or the like. In a preferred embodiment, the first colorant is a red (azo) dye (Food Red 1) sold under the name Ponceau Sx (CAS 4548-53-2), made commercially available from suppliers like Sensient Technologies, Spectrum Chemicals & Laboratory Products as well as Alfa Chem.

The second colorant suitable for use herein is limited only to the extent that the same may be used in a cosmetic composition and is a lipophilic carotenoid (i.e., coloring antioxidant) having a log P of greater than about 12.5. Illustrative non-limiting examples of the types of carotenoids suitable for use in this invention include beta-carotene, lycopene, lutein, astaxanthin, β-cryptoxanthin, mixtures thereof or the like. In a preferred embodiment, however, the carotenoid used herein is lycopene which is available from suppliers like DSM Nutritional Products and Parry Nutraceuticals.

Often, the weight ratio of first colorant:second colorant equals 0.004 to about 1,000. Preferably, the weight ratio of first colorant:second colorant is from about 1:6 to about 6:1, and most preferably, from about 1:3 to about 3:1, including all ratios subsumed therein. In an especially preferred embodiment, the total colorant (first and second colorant) used in this invention is less than about 0.4%, and preferably, less than about 0.25%, and most preferably, less than a bout 0.1%, based on total weight of the topical composition.

Optional additives may be added to the topical composition of the present invention in order to yield a desired end use composition. For example, such an end use topical composition may optionally contain a skin conditioning agent. These agents may be selected from humectants, exfoliants or emollients.

Humectants are polyhydric alcohols intended for moisturizing, reducing, scaling and stimulating removal of built-up scale from the skin. Typical polyhydric alcohols include polyalkylene glycols and more preferably alkylene polyols and their derivatives. Illustrative are propylene glycol, dipropylene glycol, polypropylene glycol, polyethylene glycol, sorbitol, glycerin, hydroxypropyl sorbitol, hexylene glycol, 1,3-butylene glycol, 1,2,6-hexanetriol, ethoxylated glycerin, propoxylated glycerin and mixtures thereof. Most preferably, the humectant is glycerin. Amounts of humectant may range (if used) anywhere from about 0.01 to 15%, preferably from about 0.01 to about 10%, optimally from about 0.75 to about 5% by weight.

Exfoliants suitable for use in the topical compositions of the present invention may be selected from alpha-hydroxycarboxylic acids, beta-hydroxycarboxylic acids and salts of these acids. Often, the exfoliants, when employed, make up from about 0.1 to about 6% by weight of the topical composition.

Most preferred are glycolic, lactic and salicylic acids and their ammonium salts.

A wide variety of C₂-C₃₀ alpha-hydroxycarboxylic acids may be employed. Suitable examples include:
α-hydroxyethanoic acid
α-hydroxypropanoic acid
α-hydroxyhexanoic acid
α-hydroxyoctanoic acid
α-hydroxydecanoic acid
α-hydroxydodecanoic acid
α-hydroxytetradecanoic acid
α-hydroxyhexadecanoic acid
α-hydroxyoctadecanoic acid
α-hydroxyeicosanoic acid
α-hydroxydocosanoic acid
α-hydroxyhexacosanoic acid, and
α-hydroxyoctacosanoic acid.

When the conditioning agent is an emollient it may be selected from hydrocarbons, fatty acids, fatty alcohols and esters, whereby the emollients (when used) typically make from about 0.1 to about 25% by weight of the total weight of the topical composition. Petrolatum is the most preferred hydrocarbon type of emollient conditioning agent. Other hydrocarbons that may be employed include mineral oil, polyolefins such as polydecene, and parafins such as isohexadecane (e.g. Permethyl 99® and Permethyl 101®).

Fatty acids and alcohols (as emollients) suitable for use often have from 10 to 30 carbon atoms. Illustrative of this category are pelargonic, lauric, myristic, palmitic, stearic, isostearic, hydroxystearic, oleic, linoleic, ricinoleic, arachidic, behenic and erucic acids and alcohols.

Oily ester emollients suitable for use in the topical compositions made according to this invention can be those selected from one or more of the following classes:
1. Triglyceride esters such as vegetable and animal fats and oils. Examples include castor oil, cocoa butter, safflower oil, cottonseed oil, corn oil, olive oil, cod liver oil, almond oil, avocado oil, palm oil, sesame oil, squalene, Kikui oil and soybean oil.
2. Acetoglyceride esters, such as acetylated monoglycerides.
3. Ethoxylated glycerides, such as ethoxylated glyceryl monostearate.
4. Alkyl esters of fatty acids having 10 to 20 carbon atoms. Methyl, isopropyl, and butyl esters of fatty acids are useful herein. Examples include hexyl laurate, isohexyl laurate, isohexyl palmitate, isopropyl palmitate, isopropyl myristate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate.
5. Alkenyl esters of fatty acids having 10 to 20 carbon atoms. Examples thereof include oleyl myristate, oleyl stearate, and oleyl oleate.
6. Ether-esters such as fatty acid esters of ethoxylated fatty alcohols.
7. Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol polyfatty esters, ethoxylated glyceryl monostearate, 1,2-butylene glycol monostearate, 1,2-butylene glycol distearate, polyoxyethylene polyol fatty acid ester, sorbitan fatty acid esters, and polyoxyethylene sorbitan fatty acid esters are satisfactory polyhydric alcohol esters.
8. Wax esters such as beeswax, spermaceti, myristyl myristate, stearyl stearate.

The topical compositions of the present invention should contain substantial levels of water. Such compositions often contain water in amounts ranging from 50 to 900%, preferably from 55 to 85% by weight, whereby the topical compositions of this invention can comprise water-in-oil or oil-in-water emulsions.

Other emollients which may be used are generally classified as siloxanes or silicon derivatives. Illustrative non-limiting examples include materials like dimethicone, cyclopentasiloxane, cross-linked siloxane based polymers and mixtures thereof.

Surfactants can be a further optional additive of the topical compositions made according to the present invention. These may be selected from nonionic, anionic, cationic or emulsifying agents. They may range, when used, in amount anywhere from about 0.1 to about 25% by weight. Illustrative nonionic surfactants are alkoxylated compounds based on C₁₀-C₂₂ fatty alcohols and acids, and sorbitan. These materials are available, for instance, from the Shell Chemical Company under the Neodol trademark. Copolymers of polyoxypropylene-polyoxyethylene, sold by the BASF Corporation under the Pluronic trademark, are sometimes also useful. Alkyl polyglycosides available from the Henkel Corporation may also be utilized for purposes of this invention.

Anionic type surfactants include fatty acid soaps, sodium lauryl sulphate, sodium lauryl ether sulphate, alkyl benzene sulphonate, mono- and di-alkyl acid phosphates and sodium fatty acyl isethionate.

Amphoteric surfactants include such materials as dialkylamine oxide and various types of betaines (such as cocoamidopropyl betaine).

Still other optional additives suitable for use in the topical compositions of this invention include thickeners. Such thickeners are often generally classified as carboxylic acid polymers, cross-linked polyacrylate polymers, polyacrylamide polymers or the like. Typical thickeners include cross linked acrylates (e.g. Carbopol 982 or Carbopol Ultrex 10), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose, polyacrylamide comprising thickeners (like Sepigel™305) and hydroxymethyl cellulose. Gums suitable for the present invention include guar, xanthan, magnesium aluminum silicate (Veegum), sclerotium, carrageenan, pectin and combinations of these gums. Amounts of the thickener may range from 0.0001 to 5%, usually from 0.001 to 1%, optimally from 0.01 to 0.5% by weight, based on total weight of the topical composition and including all ranges subsumed therein.

Typically, the optional additives used in the topical composition of this invention, collectively, make up less than about 30%, and preferably, less than about 15%, and most preferably less than about 10% by weight of the topical composition, based on total weight of the topical composition and including all ranges subsumed therein.

Optional, but especially preferred additives which may be used in the topical composition of this invention include sunscreen or ultra violet light blocking materials. Illustrative compounds are PABA, and derivatives of cinnamic and salicylic acid. For example, octyl methoxycinnamate and 2-hydroxy-4-methoxy benzophenone (also known as oxybenzone) can also be used. Octyl methoxycinnamate, 2-ethylhexyl-p-methoxycinnamate (Parsol MCX®), and 2-hydroxy-4-methoxy benzophenone are all commercially available. Others which may be used include octocrylene, butylmethoxydibenzoyl methane (Parsol 1789®) and phenylbezimidazole sulfonic acid. The preferred ultraviolet light blockers are Parsol 1789® and Parsol MCX®, and especially, a mixture of the same at a weight ratio from about 1:6 to about 6:1, and preferably, from about 1:5 to about 5:1, and most preferably, from about 1:4 to about 4:1, including all ranges subsumed therein. Typically, the amount of ultraviolet light blocker used in this invention, when desired, is from about 0.5 to about 10%, and preferably, from about 0.75 to about 6%, and most preferably, from about 1 to about 5% by weight, based on total weight of the topical composition and including all ranges subsumed therein.

Even other optional but especially preferred additives that may be used with the topical composition of this invention include physical scatterers (like TiO₂ and/or ZnO), skin lighteners like niacinamide, coumarin derived compounds 4-hydroxyphenylpyruvate, 3-propionylbenzothiazol-2-one, mixtures thereof or the like, skin care chelators (like EDTA), benefit agents like a linoleic acid, retinol and derivatives thereof or mixtures thereof, other antioxidants, like, Vitamin C, Vitamin E, and derivatives thereof (like sodium ascorbyl phosphate and tocopherol acetate), mixtures thereof or the like. When used, these other especially preferred optional additives, collectively, make up from about 0.01 to about 12%, and preferably, from about 0.1 to about 7% by weight of the topical composition, based on total weight of the topical composition and including all ranges subsumed therein.

Preservatives can desirably be incorporated into the compositions of this invention to protect against the growth of potentially harmful microorganisms, and therefore, are yet another class of optional but especially preferred additives. While it is in the aqueous phase that microorganisms tend to grow, microorganisms can also reside in the oil phase. As such, preservatives which have solubility in both water and oil are preferably employed in the present compositions. Suitable traditional preservatives are alkyl esters of para-hydroxybenzoic acid. Particularly preferred preservatives for use in this invention are methyl paraben, propyl paraben, sodium dehydroxyacetate, phenoxyethanol and benzyl alcohol. Preservatives are preferably employed in amounts ranging from 0.01% to 3% by weight of the topical composition. In an especially preferred embodiment, the preservative employed is methyl paraben, propyl paraben or a mixture thereof, and the weight ratio of preservative to ultraviolet light blocker employed is from about 1:8 to about 8:1, and preferably, from about 1:6 to about 6:1, and most preferably, from about 1:4 to about 4:1, including all ratios subsumed therein.

Minor adjunct ingredients may also be included such as fragrances, antifoam agents, and colorants, each in their effective amounts to accomplish their respective functions.

When making the compositions of the present invention, the desired ingredients can be mixed, in no particular order, and usually at temperatures from about ambient to about 65° C. and under atmospheric pressure. In a preferred embodiment, however, water is added to oil.

The viscosity of the topical composition prepared according to this invention is typically from about 2,000 to about 400,000 cps, and preferably from about 3,000 to about 300,000 cps, and most preferably, from about 5,000 to about 225,000 cps taken at a shear rate of 1S⁻¹ at ambient temperature with a strain controlled parallel plate rheometer (like those sold by T.A. Instruments under the Ares name).

The colorants used herein may be encapsulated (either or both, if desired) by conventional techniques. Such techniques include the use of cyclodextrin, whereby the conventional technique is further described in Eur. J. Org. Chem. 2005, pages 4051-4059.

When applying the topical composition of this invention, the consumer is typically directed to use approximately 0.1 g of composition for about every 5 cm² of skin in order to yield a healthy and desirable skin color.

The packaging for the compositions of this invention is not limited and can include a bottle, tube, foil wrap, roll-ball applicator, squeeze container or lidded jar.

The examples below are provided to illustrate the invention and are not intended to limit the scope of the claims.

### Example 1

Topical compositions, according to this invention, were made by combining the following ingredients:

| Ingredient | Weight Percent in Formula |
|---|---|
| Humectant | 0.1-3.0 |
| Surfactant | 0.0-20 |
| First colorant* | .0001-.0003 |
| Emollient | 2-5 |
| Thickener | 0.0-0.3 |
| Preservative** | 0.2-0.5 |
| Physical scatterer | 0.3-1 |
| Antioxidant (second colorant)*** | 0.001-0.002 |
| Skin lightener**** | 1-4 |
| Sunscreen/UV absorber | 1-2 |
| Fragrance | 0.2-0.4 |
| Vitamin C | 0.0-0.3 |
| Vitamin E | 0.2-1.2 |
| Water | Balance |

*Food Red 1 (Ponceau SX)
**Methyl and Propyl Paraben
***Lycopene 10% mixture from DSM Nutritional Products
****Niacinamide

### Example 2

Topical compositions similar to those made in Example 1 were stored for about three (3) months at ambient temperature. Unexpectedly, the color of the compositions in packaging and when applied (after the three (3) month period) remained substantially the same as the color when the compositions were first made. The topical compositions when made and after storage satisfied the L* and hue characteristics described herein.

What is claimed is:

1. A consumer grade colorant comprising:
(a) a first colorant comprising dye, pigment or both; and
(b) a second colorant comprising a lipophilic carotenoid having a log P of greater than about 12.5;
wherein the weight ratio of first colorant (a) to second colorant (b) satisfies
a/b=about 0.004 to about 1,000.

2. The consumer grade colorant according to claim 1 wherein the colorant consists entirely of the first colorant and second colorant.

3. The consumer grade colorant according to claim 1 wherein the lipophilic caretenoid is beta-carotene, lycopene, lutein, astaxanthin, β-cryptoxanthin or a mixture thereof.

4. A topical composition influencing skin color comprising:
(a) a first colorant comprising dye, pigment or both; and
(b) a second colorant;
the second colorant being an antioxidant and comprising a lipophilic carotenoid having a log P of greater than about 12.5 where the composition has a L* value from about 60 to about 90 and a hue value from about −50 to about 50 for at least about three (3) months when stored at about ambient temperature and further where the weight ratio of the first colorant (a) to second colorant (b) satisfies:
a/b=about 0.004 to about 1,000.

5. The topical composition according to claim 4 wherein the first colorant is Red Pigment 4, Food Red 1, Acid Orange 7, Pigment Red 57, Food Yellow 3, Acid Red 33, Food Red 12, Acid Yellow 23, Food Yellow 4, Food Green 3, Food Blue 2, Acid Blue 9, Solvent Red 43, Acid Red 87, Solvent Red 48, Acid Red 92, Solvent Violet 13, Acid Violet 43, Solvent Green 3, Acid Green 25, Vat Red 1, Pigment Metal 1, Pigment Blue 29, Pigment White 21, Pigment White 22, Pigment White 14, Pigment White 31, Pigment Brown 6, or Pigment Red 101, Pigment 102, Pigment Brown 6, Pigment Yellow 42, Pigment Yellow 43, Pigment Black 11, Pigment White 18, Pigment Violet 16, Pigment White 6, Pigment White 4 or a mixture thereof.

6. The topical composition according to claim 4 wherein the second colorant is beta-carotene, lycopene, lutein, astaxanthin, β-cryptoxanthin, or a mixture thereof.

7. The topical composition according to claim 4 wherein the weight ratio of first colorant to second colorant is from about 1:6 to about 6:1.

8. The topical composition according to claim 4 wherein the weight ratio of first colorant to second colorant is from about 1:3 to about 3:1.

9. The topical composition according to claim 4 wherein the first colorant consists essentially of red dye and the second colorant consists essentially of lycopene.

10. The topical composition according to claim 4 wherein the topical composition is substantially free of formaldehyde releasing preservative.

11. The topical composition according to claim 4 wherein the topical composition further comprises physical scatterers.

12. The topical composition according to claim 4 wherein the topical composition further comprises a preservative which comprises methylparaben, propylparaben or both, and an ultraviolet light blocker comprising butylmethoxydibenxoyl methane and 2-ethylhexyl-p-methoxycinnamate.

13. The topical composition according to claim 12 wherein the topical composition comprises from about 0.01 to about 3% by weight preservative and from about 0.5 to about 6% by weight ultraviolet light blocker.

14. The topical composition according to claim 4 wherein the first colorant, the second colorant or both are encapsulated.

15. The topical composition according to claim 13 wherein the preservative and ultraviolet light blocker are at a weight ratio form about 1:8 to about 8:1.

16. The topical composition according to claim 4 wherein the composition further comprises a skin lightener.

17. The topical composition according to claim 16 wherein the skin lightener is niacinamide.

18. A method for influencing skin color of a consumer comprising the step of applying the topical composition of claim 4 to skin on the consumer.
